SEQUENCE LISTING (2 sequences)

SEQ ID NO: 1 - DNA, 47 bp, Artificial sequence
  monomer of the site for binding to TLR9
gcgcgctgcgcgctgcgcgcgcatgcgcgcgctgcgcgctgcgcgct

SEQ ID NO: 2 - protein, 31 aa, Artificial sequence
  modified beta-endorphin
YGGFMTSEKSQTPLVTVFKNAVVKNAYKKGE

(19) United States
(12) Patent Application Publication
(10) Pub. No.: US 2021/0077583 A1
(43) Pub. Date: Mar. 18, 2021

(54) PLASMID DNA ENCODING BETA-ENDORPHIN, BACTERIAL PRODUCER, ANALGESIC AGENT

(71) Applicant: Ilya Vladimirovich DUKHOVLINOV, St. Petersburg (RU)

(72) Inventor: Ilya Vladimirovich DUKHOVLINOV, St. Petersburg (RU)

(21) Appl. No.: 16/766,836

(22) PCT Filed: Oct. 17, 2018
(86) PCT No.: PCT/RU2018/050128

§ 371 Date: Oct. 3, 2020

(30) Foreign Application Priority Data
Nov. 26, 2017 (EA) 201792364

(51) Int. Cl. A61K 38/22; C12N 15/74

(52) U.S. Cl. CPC A61K 38/22; C12N 15/74

(57) ABSTRACT

Inventions relate to medicine, pharmacology, biotechnology, molecular biology, genetic engineering and can be used for analgesia. A plasmid DNA for transient expression in mammalian cells is proposed and represented by DNA backbone containing prokaryotic and eukaryotic elements, as well as a fragment providing enhanced capture of plasmid DNA by cells and a polynucleotide encoding beta-endorphin modified for increasing the affinity for receptors and codon-optimized for expression in mammalian cells. There are also proposed a producer of such plasmid DNA on the basis of a bacterial cell and an analgesic agent for application in mammals, in particular, humans, on its basis. The technical result of the use of the developed plasmid DNA and analgesic based on it is to increase the controllability of the synthesis of beta-endorphin exactly, in increasing the efficiency of plasmid DNA from which beta-endorphin is synthesized, and reducing its amount to achieve analgesia, in increasing the duration of analgesia and in expanding the spectrum of analgesic drugs.

### TECHNICAL FIELD

Inventions relate to medicine, pharmacology, biotechnology, molecular biology, genetic engineering and can be used for analgesia.

### BACKGROUND ART

It is important to recognize that pain is not just an indicator of the underlying disease or process of injury, but an independent problem that causes great damage to individuals and society as a whole. In order to improve the quality of life, pain relief per se should be a therapeutic goal.

It is shown that the change in the concentration of beta-endorphin in the blood plasma is directly dependent on the type of pain syndrome, its intensity and the effectiveness of analgesia [Beta-endorphin—a marker of the effectiveness of pain relief in acute pain and chronic pain syndrome in cancer patients/Z. V. Pavlova [and others]//Problems of clinical medicine.—2007.—N1. P. 36-40.—ISSN 1817-8359]. This endogenous opioid is able to reduce pain. However, the introduction of endogenous opioids, or molecules based on them, can be complicated by the formation of antibodies to them, which can lead to side effects.

There are known recombinant viruses that carry the endorphin precursor gene or endorphin.

A retrovirus-based pRetro-Off-POMC vector encoding proopiomelanocortin (POMC), a precursor of beta-endorphin, is known for the synthesis of beta-endorphin in the absence of doxycycline [https://doi.org/10.1016/S1525-0016(16)40805-1].

However, the delivery of the gene into the organism using the viral vector has a number of significant drawbacks associated with the risk of infection and specific reactions to the vector of such nature in principle—for example, an inflammatory reaction may occur, which eliminates the re-introduction of the vector. Obtaining such a vector carries the risks of getting into the body of substances that can cause various undesirable effects, or is complex and not cost-effective. Also, viral vectors have the ability to replicate, which reduces the degree of control over the expression of the target protein, which, in turn, is not always desirable and applicable, especially with regard to analgesia.

Plasmid DNA is a safer means of delivering the target gene to the organism. It is shown that plasmids are maintained as episome and do not integrate into the genome, the protein is synthesized from them and then secreted from the cell, as a result, nonimmunogenic, safe use of this protein for analgesia is carried out. The protein synthesized in the organism from plasmid DNA undergoes natural post-translational processing, and the correct folding of the protein due to cellular chaperones is ensured. These modifications are difficult to achieve in the production of proteins, which can dramatically affect a number of their functions. The natural mechanisms of metabolism and catabolism of the active substance are used, without the formation of toxic products, due to the nature of the plasmid construct and the protein encoded by it. Plasmid DNA does not replicate after being introduced into the body of a mammal, which allows control over the amount of protein synthesized, and, accordingly, over analgesia.

It should also be noted that the production, purification and storage of DNA preparations is economically more profitable than of protein ones, because they are more stable, they can be produced in large quantities and at lower cost.

The following plasmid DNAs encoding beta endorphin are known.

For the implementation of controlled antinociceptive gene therapy, a system of three plasmids induced by tetracycline, encoding beta-endorphin, a tetracycline activator of transcription and a tetracycline silencer of transcription is introduced (PMID:18064731). The introduction of plasmids by electric transfer was carried out in the spinal cord. Doxycycline for the regulation of endorphin expression was administered intraperitoneally. The expression of endorphin was carried out only with the introduction of doxycycline. It is also proposed to introduce a system of three plasmids into the cavity of the spinal canal, to relieve pain in the extremities, caused by compression of the sciatic nerve. However, it should be noted that the synthesis of endorphin in this case is controlled by the synthesis of a transcription activator, which, in turn, is synthesized under the influence of doxycycline, so this system is difficult to control. It would also be more preferable to use a safer way of introduction. Fewer variants of injected plasmids is also preferred, as it reduces the immunogenicity of the injected DNA drug and reduces the risk that some component of the system will not work.

A system is known of two plasmids encoding POMC and silencer controlled by doxycycline, which is also introduced into the spinal cord by electric transfer [PMID:15116065]. However, the disadvantages are similar to the system described above.

There is a known a remedy for reducing or suppressing pain in higher animals, including humans, based on one of the variants of expression constructs containing CMV promoter; gene encoding POMC or POMC, in which the fragments encoding ACTH and β-MSH are replaced by beta-endorphin; polyadenylation site [EP1363658 (A2)]. Constructs can be circular or linear. MIDGES additionally contain intron [cf. EP 0 941 318 B1]. Vectors for obtaining such expression constructs are also known-plasmids pMOK and pNOK, which contain in one of the variants a gene encoding beta-endorphin in different variants. However, for administration to the animal, constructs containing a sequence encoding POMC are used, as described above, not beta-endorphin directly. Introduction is carried out locally by injection.

A later article showed that MIDGE (Non-viral, non-plasmid minimalistic, immunologically defined gene expression vectors) carrying POMC-coding gene, did not demonstrate an analgesic effect [PMID:20003437].

We propose a plasmid DNA that carries a nucleotide sequence encoding beta-endorphin directly, with introduced mutations for greater receptor affinity, codon-optimized for expression in mammalian cells, with introduced heterologous secretory sequence, and also a fragment providing strong binding to Toll-like receptor 9 (TLR9) for greater transformation of cells by plasmid DNA. Such plasmid DNA, an analgesic based on it can be injected locally or systematically.

The technical result of the use of the developed plasmid DNA and analgesic based on it is to increase the controllability of beta-endorphin synthesis by introducing a vector that carries the sequence of beta-endorphin directly, not a precursor which is processed differently in different tissues.

The technical result is also in increasing the efficiency of plasmid DNA, from which beta-endorphin is synthesized, and in reducing its quantity to achieve analgesia. These technical results are achieved due to the greater affinity of the synthesized beta-endorphin to the receptor due to specific mutations introduced in the gene encoding it. They are also achieved by more efficient cell transformation due to the presence of a fragment in the plasmid DNA that provides strong binding to TLR. These technical results are achieved by optimizing the codon composition of the nucleotide sequence encoding beta-endorphin for expression in mammalian cells, as well as due to the fact that the plasmid DNA contains elements that cause the stability of mRNA and, accordingly, increase the half-life of mRNA, as a result the protein synthesis from one mRNA molecule is carried out more times, and also as a result, the amount of synthesized protein increases; so protein synthesis is more intense.

In addition, the technical result is to increase the duration of analgesia and is achieved by the fact that the nucleotide sequence encoding beta-endorphin contains elements that cause the stability of mRNA and, accordingly, increase the half-life of mRNA, as a result the protein synthesis from one mRNA molecule is carried out more times, which leads to an increase in the amount of synthesized proteins; and also is achieved by optimizing the codon composition of the nucleotide sequence encoding beta-endorphin for expression in mammalian cells, as a result the protein synthesis is more intensive.

When introduced into practice, this will allow to significantly reduce the amount of injected plasmid DNA (10-50 times) in comparison with the doses currently used in the national and world practice in gene therapy.

The technical result is also in the expansion of the analgesic drugs spectrum. When poor tolerability or intolerance to analogs, the proposed analgesic will allow to carry out analgesia, due to which a patient will be able to perform actions that he couldn't or didn't want to do without analgesia, for example, to dare on a required procedure, as well as improve the quality of life, for example, will receive less unpleasant sensations, than without the use of an analgesic, or will relieve acute or chronic pain. This technical result is achieved by using analgesic of the present invention.

The technical result from the use of the producer of the developed plasmid DNA is in obtaining such plasmid DNA.

According to the time characteristics, there are two types of pain:
acute pain—a new, recent pain that is inextricably bound with an injury that has caused it and, as a rule, is a symptom of a disease and disappears when the injury is eliminated [Eddy N. B., Leimbach D. J.//Pharmacol Exp Ther.—March; 107(3):385-93. —1953] (including pre- and postoperative, post-traumatic, related to burns, acute pain during childbirth, pain caused by spinal cord injury, acute headache, pain related to HIV/AIDS, sickle cell crisis, trigeminal neuralgia, pancreatitis and other pains in the gastrointestinal tract, related to myocardial infarction and other major cardiac events, interventional pain (in diagnostic and therapeutic procedures), acute during chronic pain [WHO Normative Guidelines on Pain Management, Geneva, June 2007], which lasts up to 2-3 months, and can irradiate, and
chronic pain—lasting for a long period of time (over 2-3 months), even after the cause has been eliminated, often acquires the status of an independent disease, for example, an inflammatory process [Eddy N. B., Leimbach D. J.//Pharmacol Exp Ther. —March; 107(3):385-93.—1953], with a decrease in the effectiveness of analgesics observed. A chronic pain may be a chronic pain in malignant diseases (including pain in cancer patients, HIV/AIDS, related to amyotrophic lateral sclerosis (ALS), multiple sclerosis, end-stage of an organ failure, advanced chronic obstructive pulmonary disease, advanced congestive heart failure, parkinsonism) and chronic pain not associated with a malignant disease (chronic musculoskeletal pains, such as back pain or lower back pain, in a chronic degenerative arthritis, osteoarthritis, rheumatoid arthritis, myofascial and rheumatic pains, a chronic headache, migraine, pain in the bones; neuropathic pain (including pain related to nerve compression, trauma, pain after nerve damage and amputation), related to diabetic neuropathy, complex regional pain syndromes (type I and type II), related to spasms of skeletal muscles, postherpetic neuralgia, chronic pain after surgical interventions; visceral pain (related to stretching the viscera and colic); and chronic pain related to sickle cell anemia [WHO Normative Guidelines on Pain Management, Geneva, June 2007].

With the use of the proposed analgesic, it is possible to relieve and neutralize both types of pain described above.

### SUMMARY OF INVENTION

A plasmid DNA for transient expression in mammalian cells is proposed represented by a backbone containing prokaryotic elements, an origin of replication and a marker gene, and eukaryotic elements, a strong promoter, an mRNA leader sequence, as well as regulatory sequences for these elements, at least one site for cloning a gene of interest and at least one site for at least one primer annealing for analysis of plasmid DNA composition, by a fragment, characterized by SEQ ID NO: 1, repeated tenfold consecutively, located between any two elements of the plasmid DNA, and by a polynucleotide represented by a heterologous secretory sequence, a fragment encoding a modified beta-endorphin, characterized by SEQ ID NO: 2, codon composition of last two elements is optimized for expression in mammalian cells, and a termination sequence.

Also there are given a producer of such plasmid DNA based on a bacterial cell and an analgesic agent based on such plasmid DNA in an effective amount, also containing a pharmaceutically acceptable excipient, for use in mammals, in particular, in humans.

### DESCRIPTION OF EMBODIMENTS

Plasmid DNA contains elements essential for organisms of its maintaining and use, together with the corresponding regulatory sequences. Regulatory sequences are nucleotide sequences that can affect gene expression at the transcription and/or translation level, as well as mechanisms that ensure the existence and maintenance of plasmid DNA.

Plasmid DNA production is economically most beneficial to carry out in prokaryotic cells, mainly bacterial cells. In this regard, the plasmid DNA of the present invention contains elements for maintenance and amplification, mainly in large quantities, in bacterial cells. Such essential elements are a bacterial origin of replication for maintenance in a cell with a medium, preferably a high number of copies per cell, and a marker gene for selection of a producer strain. Suitable origin of replication is represented by pM1 (der.), ColE1 (der.) and F1, pUC and F1, but is not limited to them. A suitable marker gene is represented by a reporter gene or a gene of resistance to an antibiotic, for example, to ampicillin, mainly kanamycin, but is not limited to them.

Plasmid DNA according to the present invention contains elements for effective functioning in mammalian cells.

Such an element is a promoter with the corresponding regulatory sequences of natural promoters with their own regulatory elements. (CaM kinase II, CMV, nestin, L7, BDNF, NF, MBP, NSE, p-globin, GFAP, GAP43, tyrosine hydroxylase, kainate receptor subunit 1 and glutamate receptor subunit B, and others), or synthetic promoters with regulatory sequences to obtain the necessary character of expression (the ratio of duration and level of expression) of the target gene at the transcription level.

The promoter is an important component of the plasmid that triggers the expression of the gene of interest. Human CMV/immediate-early or CMV-chicken-β actin (CAGG) promoter are classic promoters for plasmid DNA components of drugs. CMV promoters are used for most DNA vaccines, as they mediate high levels of constitutive expression in a wide range of mammalian tissues. [Manthorpe M, Cornefert-Jensen F, Hartikka J, et al. Gene therapy by intramuscular injection of plasmid DNA: studies on firefly luciferase gene expression in mice. Hum. Gene Ther. 1993; 4(4):419-431] and do not inhibit the downstream reading. An increase in the expression level is observed when the CMV promoter is changed, for example, by including HTLV—1R-U5 downstream from the cytomegalovirus promoter or when using the SV40-CMV chimeric promoter [Williams J A, Carnes A E, Hodgson C P. Plasmid DNA vaccine vector design: impact on efficacy, safety and upstream production. Biotechnol. Adv. 2009; 27(4):353-370]. Tissue-specific host promoters that allow to avoid constitutive expression of antigens in inappropriate tissues, which generally leads to a decrease in immunogenicity [Cazeaux N, Bennasser Y, Vidal P L, Li Z, Paulin D, Bahraoui E. Comparative study of immune responses induced after immunization with plasmids encoding the HIV-1 Nef protein under the control of the CMV-IE or the muscle-specific desmin promoter. Vaccine. 2002; 20(27-28):3322-3331], are alternative to CMV promoters.

Possible regulatory sequences in relation to the promoter:
enhancer, to increase expression level through improvement of RNA polymerase and DNA interaction,
insulator, for modulating enhancer functions,
silencers, or their fragments, to reduce the transcription level, for example, for tissue-specific expression,
5′ noncoding region upstream of the promoter, including intron.

The plasmid DNA of the present invention contains at least one of the above regulatory sequences, depending on the plasmid DNA variant based on the choice of promoter and the desired expression parameters of the targeted gene. Based on the existing level of technology, the well-known and obvious variations of such elements and their use, the plasmid DNA according to the present invention may contain any combinations that meet the above conditions, in which beta-endorphin is synthesized in mammalian cells from plasmid DNA. Using a silencer or an insulator as part of the construction, it is possible to regulate the expression of the target gene, the gene of the described beta-endorphin, that means the control over the amount of the synthesized protein, and, in principle, over protein synthesis in itself, and, accordingly, over analgesia: if necessary, it is possible to stop or reduce the expression of the gene in a short time. In the latter case, it is possible to perform tissue-specific expression if necessary.

Other regulatory sequences:
noncoding region downstream from the promoter, including intron, to increase mRNA stability and the target gene expression.

In one of the variants, the plasmid DNA of the present invention additionally contains such regulatory element.

Plasmid DNA according to the present invention also contains such an important element as mRNA leader sequence containing the Kozak sequence directly before ATG start codon which allows to increase expression [Kozak M. Recognition of AUG and alternative initiator codons is augmented by G in position +4 but is not generally affected by the nucleotides in positions +5 and +6. EMBO J. 1997; 16(9):2482-2492].

Plasmid DNA also contains a site, preferably sites, different, for cloning of a targeted gene, for correct orientation of a target gene in a plasmid DNA, and a site, preferably sites, for primers annealing for its sequencing.

Plasmid DNA also contains a termination sequence, which is necessary for mRNA stability preservation, proper transcription termination and export of mRNA from the nucleus. Gene expression can be affected by changing the termination sequence, including its shortening. Plasmid DNA also contains a termination sequence containing sequentially a stop codon, the 3′ noncoding region with signal and polyadenylation site, stop codon, with the help of which mRNA remains stable, and the proper transcription termination and export of mRNA from the nucleus are performed.

Polyadenylation (polyA) is necessary to stabilize the transcript. PolyA sequence change may lead to an increase in the level of gene expression. [Norman J A, Hobart P, Manthorpe M, Felgner P, Wheeler C. Development of improved vectors for DNA-based immunization and other gene therapy applications. Vaccine. 1997; 15(8):801-803]. In pVAX1 plasmid (Invitrogen, Carlsbad, Calif.), the bovine growth hormone terminator region contains a homopurine region that is sensitive to nuclease. It has been shown that an alternative polyA sequence can significantly improve plasmid stability to nuclease [Azzoni A R, Ribeiro S C, Monteiro G A, Prazeres DMF. The impact of polyadenylation signals on plasmid nuclease-resistance and transgene expression. J Gene Med. 2007; 9:392-402]. The introduction of two stop codons allows increasing the efficiency of the transcription terminator.

The terminating sequence of the proposed plasmid DNA is represented by a native, i.e. inherent from the target gene, or another one, stronger, which is represented, for example, by the terminating sequence of bovine growth hormone (BGH), but it is not limited to such, and in the second variant it may contain an additional stop codon before the 3′ noncoding region.

Based on the existing state of art, on known and obvious variants of such an element, plasmid DNA according to the present invention can contain any terminating sequence that meets the above conditions, in the presence of which the synthesis of beta-endorphin is performed in mammalian cells from the plasmid DNA.

Plasmid DNA contains a fragment characterized by SEQ ID NO: 1, repeated tenfold consecutively, which is located between any two elements of the plasmid DNA, thereby not interfering with the functioning of any of its elements. This fragment provides enhanced uptake of the plasmid DNA by cells due to its tropism to the Toll-like Receptor 9 (TLR9).

Plasmid DNA also contains a heterologous secretory sequence optimized in codon composition for expression in mammalian cells. In one embodiment of the invention, plasmid DNA contains, for example, TPA (tissue-type plasminogen activator isoform 1 preproprotein [Homo sapiens], NCBI Reference Sequence: NP_000921.1) secretory sequence, but is not limited to such. The advantage of using TPA secretory sequence is in extensive previous clinical experience, as well as in its high performance demonstrated in relation to the expression of a secreted protein from different target genes.

Plasmid DNA also contains a fragment encoding beta-endorphin modified to increase receptor affinity, characterized by SEQ ID NO: 2, codon-optimized for expression in mammalian cells.

Codon-optimization was carried out to increase the expression of the targeted gene by increasing the efficiency of information reading from this mRNA on the ribosomes. It can be carried out manually or using a specialized software, for example, on the site molbiol.ru, on the base of amino acid sequence of the protein.

The plasmid DNA may also additionally contain elements initial for cDNA of the indicated gene, determining the stability of this mRNA, such as the termination sequence (3′ noncoding region containing the signal and polyadenylation site, as well as the transcription termination signal—stop codon). Accordingly, in this case similar elements are absent in the backbone of the plasmid DNA, or they do not function.

Plasmid DNA for the delivery of the gene causing analgesia, combines such properties of a DNA vaccine based on a plasmid DNA, as a high level of expression of the gene of interest in mammalian cells, and such properties of the vector for gene therapy as the lack of immunogenicity and duration of gene expression, however, for example, by increasing mRNA stability. Such DNA does not integrate into the genome and does not replicate in mammalian cells.

Suitable vectors for expression in mammalian cells are represented by those known to a person averagely skilled in the art and described in the literature [Hartikka J, Sawdey M, Cornefert-Jensen F, Margalith M, Barnhart K, Nolasco M, Vahlsing H L, Meek J, Marquet M, Hobart P, Norman J, Manthorpe M. An improved plasmid DNA expression vector for direct injection into skeletal muscle. Hum Gene Ther. 1996 Jun. 20; 7(10):1205-17 and others], as well as plasmids that can be created by an average specialist in the field using the recommendations on the elements of vectors [“Cloning Vectors”, ed. Pouwls et al., Elsevier, Amsterdam-New York-Oxford, 1985, ISBN 0 444 904018, Williams J A, Carnes A E, Hodgson C P. Plasmid DNA vaccine vector design: impact on efficacy, safety and upstream production. Biotechnol Adv. 2009 July-August; 27(4):353-70. doi: 10.1016/j.biotechadv.2009.02.003. Epub 2009 Feb. 20. Review and others]. The preferred plasmid DNA for use in humans are vectors tested on humans containing the above-described elements with the corresponding regulatory sequences, feasibly modified to match the stated criteria, which allows to reduce the number of required studies for drug registration. However, it is possible to use other plasmid DNAs containing the required described elements. For plasmid DNA for use in mammals, except for humans, the requirements are less stringent, and therefore it is possible to use a wider range of plasmids.

The sequence of the described elements in the plasmid DNA is clear to the average specialist in this field.

A producer of the plasmid DNA is given based on a bacterial cell (on the basis of cells, mainly, Escherichia coli, but not limited to them). It is clear to the average specialist in this field that, using plasmid DNA according to the invention and a bacterial cell, for example, commercial, but not limited to it, it is possible to create a producer of plasmid DNA, for example, by standard methods, for example, transfection, electroporation or a gun with particles. To reduce the probability of mutations due to methylation of plasmid DNA, it is preferable to use a strain of a microorganism that does not contain methylase in the genome.

Also an analgesic agent is given based on the characterized plasmid DNA, in an effective amount, also containing a pharmaceutically acceptable excipient, for use in mammals, in particular, in humans. Pharmaceutically acceptable carriers or buffer solutions are known from the state of the art and include those described in various texts, such as Remington's Pharmaceutical Sciences.

This pharmaceutical composition is intended for the treatment, relief and/or prevention of pain, in particular acute or chronic pain, sensitivity disorders.

It is also possible to use other technologies of delivery of plasmid DNA into cells of various tissues, for example, a needle-free syringe, which allows delivering into living animals cells of different tissues [Furth et al., Analytical Biochemistry, 205, 365-368, (1992)].

It has been shown that muscle fibers do not normally express MHC antigens, however, in inflammation associated with infection or in the presence of interferon gamma muscle fibers are able to produce antigens in the complex with MHC of the first class, or even of the second class [MECKERT, P. C, HONTEBEYRIE-JOSKOWICZ, M., CHAMBO, J., LEVIN, M., and LAGUENS, R. P. (1991). Trypanosoma cruzi: Aberrant expression of class II major histocompatibility complex molecules in skeletal and heart muscle cells of chronically infected mice. Exp. Parasitol. 72, 8-14; Hohlfeld and ENGEL, A. G. (1984) The immunobiology of muscle. Immunol. Today 15, 269-274; MANTEGAZZA, R., and BERNASCONI, P. (1994). Cellular aspects of myositis. Curr. Opin. Rheumatol. 6, 568-574, Hartikka J, Sawdey M, Cornefert-Jensen F, Margalith M, Barnhart K, Nolasco M, Vahlsing H L, Meek J, Marquet M, Hobart P, Norman J, Manthorpe M. An improved plasmid DNA expression vector for direct injection into skeletal muscle. Hum Gene Ther. 1996 Jun. 20; 7(10):1205-17], therefore, it is preferable to introduce plasmid DNA in such a way when muscle injury is minimized.

The authors of the present invention have conducted laboratory studies confirming the possibility of implementing a group of characterized inventions. The results of the research are illustrated with examples (1-3).

### EXAMPLES

### Example 1. Obtaining Plasmid DNA Encoding the Modified Beta-Endorphin

1.1. The gene nucleotide sequence was calculated which is collinear for the amino acid sequence of the beta-endorphin encoded by it modified for greater affinity for the receptors, characterized by SEQ ID NO: 2, with flanking the target gene by restriction sites, as well as adding Kozak sequence before the start codon to initiate translation, after the start codon—signal sequence, for example, TPA (mdamkrglccvlllcgavfvsps), either represented by a.a. MLLLLLLLLLLALALA, for secretion of the synthesized protein from an eukaryotic cell, with simultaneous codon-optimization for expression in human cells using a specialized software on the site molbiol.ru.

The calculated nucleotide sequence was synthesized chemically using DNA synthesizer ASM-800 (BIOSSET, Russia).

1.2. A chemical synthesis was performed of DNA characterized by SEQ ID NO: 1, repeated tenfold sequentially, with flanking of such DNA by restriction sites for cloning in plasmid DNA.

1.3. The synthesized DNA fragments were cloned into pVAX1 (Invitrogen), pVR1012 (Vical), pcDNA3.1(+) (Invitrogen) plasmids. Also pcDNA3.1(+) vector was obtained incapable of neomycin expression due to restriction of this vector with the restriction enzyme NsiI, in the region of the SV40 promoter (−71 b.p.). In the resulting vector, the obtained fragments were also cloned. A fragment that causes TLR9 tropism was placed between different elements of plasmid DNA.

1.4. For the ligation reaction, 3 μl of the synthesized DNA solution, 1 μl of the prepared vector solution, 5 μl of ligation buffer ×2 and 1 μl of T4 ligase were taken. The reaction was carried out at +20° C. for 2 hours.

After that, the mixture was heated at +95° C. for 10 min and purified from salts by dialysis on nitrocellulose filters with a pore diameter of 0.025 μm (Millipore, USA). Dialysis was performed against a solution containing 0.5 mM EDTA in 10% glycerol for 10 minutes.

1.5. Then, E. coli cells of the strain DH10B/R (F-mcrA, Δ(mrr-hsdRMS-mcrBC), φ80dlacZΔM 15, ΔlacX74, deoR, recA1, endA1, araD139, Δ(ara,leu)769, galU, galKλ-, rpsL, nupG), or XL1-Blue (recA1 endA1 gyrA96 thi-1 hsdR17 supE44 relA1 lac [F′ proAB lacIqZΔM15 Tn10 (Tetr)) were transformed with the obtained plasmid DNA by electroporation using MicroPulser (BioRad) electroporator. This strain does not contain methylase, which minimizes the possibility of mutations occurrence in DNA, including in the gene cloned in the plasmid maintained in this strain. 1 μl of the dialyzed ligase mixture was added to 12 μl of competent cells, placed between the electrodes of electroporation unit, and processed by a current pulse.

After transformation, the cells were placed in 1 ml of SOC-medium (2% bactotryptone, 0.5% yeast extract, 10 mM NaCl, 2.5 mM KCl, 10 mM MgCl2, 10 mM MgSO4, 20 mM glucose) and incubated for 40 min at +37° C.

1.6. The clones of E. coli cells containing the obtained plasmid DNA were detected on a selective medium containing LB agar, 50 μg/ml of kanamycin, or ampicillin (for plasmid DNA based on pcDNA3.1(+)).

Plasmid DNA was isolated from the grown clones. Isolation of the plasmid DNA was performed using Wizard Minipreps DNA Purification System (Promega, USA). Purified recombinant plasmid DNA was verified by sequencing.

1.7. Sequencing of cloned fragments was performed by the Sanger method using a set of Applied Biosystems BigDye® Terminator (BDT) v3.1 Cycle Sequencing Kit (Applied Biosystems, USA) according to the attached manual. For the reaction products labeling fluorescent dye-labeled ddNTP were used, each ddNTP corresponding to its own dye. Unlabeled plasmid-specific primers were used for sequencing. A PCR reaction was carried out, then the reaction mixture was purified from free labeled ddNTP using instructions to the BigDye x-Terminator Purification Kit (Applied Biosystems, USA) and the sequencing reaction products were separated using capillary sequencer Applied Biosystems 3500/3500xL Genetic Analyzer (Applied Biosystems, USA) and reagent 3500/3500xL Genetic Analyzer Polymer “POP-6™” (Applied Biosystems Biosystems, United States).

The results of sequencing reaction products separation were recorded by scanning with laser and detection of four fluorescent dyes contained in all types of ddNTP.

1.8. Computer analysis of DNA sequences was performed using a personal computer using Chromas and BioEdit programs. The nucleotide sequences of the DNA fragments studied were aligned with the calculated ones, the identity of the synthesized fragments to the calculated ones was demonstrated. As a result, clones of E. coli cells were selected containing the full-length sequences of targeted genes in the plasmids.

### Example 2. Obtaining the Plasmid DNA Encoding Modified Beta-Endorphin for Conducting of the Trial on Laboratory Animals

A separate colony of E. coli cells of plasmid DNA producer strain grown on LB-agar in a Petri dish with the addition of kanamycin (or ampicillin), was placed in 10 ml of selective medium. Cells were grown overnight at +37° C. under constant stirring conditions (250 rpm). The obtained cells were collected by centrifugation at 4000 g. Further isolation and purification of plasmid DNA was performed using EndoFree Plasmid Mega Kit (Qiagen), which allows to obtain pyrogen-free DNA. The isolated plasmid DNA was analyzed by electrophoresis in a 0.8% agarose gel, its concentration was measured using fluorimetry.

As a control solution, water was used without adding the test preparation. 1.950 ml of water and 0.05 ml of the test solution were placed in a 2 ml optical density measurement unit, mixed, and the optical density was measured at a wavelength of 260 nm. DNA concentration was determined by the formula:

C(μg/ml)=40A₂₆₀K,

where A260 is the optical density of the preparation measured at a wavelength of 260 nm; K (μg/ml)—for DNA 50 μg/ml (50 μg/ml of double-stranded DNA in water); 40 dilution of the tested preparation.

As a result, it was determined that plasmid DNA was obtained with a concentration of 3-5 mg/ml. The yield of plasmid DNA ranged from 3.5 mg to 5 mg from 11 of the culture medium.

The purity of the obtained preparation of plasmid DNA was judged by the ratio of the optical density of the preparation, measured at a wavelength of 260 nm, to the optical density of the preparation, measured at a wavelength of 280 nm (A₂₆₀/A₂₈₀) and the ratio of the optical density of the preparation, measured at a wavelength of 260 nm to the optical density of the preparation, measured at a wavelength of 230 nm (A₂₆₀/A₂₃₀). The measurements were carried out in an aqueous solution; water was used as a control solution without the addition of the test preparation.

Pure DNA preparations are characterized by the ratio of A₂₆₀/A₂₈₀>1.80 and A₂₆₀/A₂₃₀>1.80. The values determined in the experiment corresponded to the values of the ratios A₂₆₀/A₂₈₀ and A₂₆₀/A₂₃₀ for pure preparations, for all the obtained preparations of plasmid DNA.

Also, quantitative determination of protein impurities in the obtained plasmid DNA preparations was performed using the microBCA assay [Smith, P. K., et al., Measurement of protein using bicinchoninic acid. Analyt. Biochem. 150, 76-85 (1985)] by measuring the optical density of the resulting coloured protein complexes with copper and bicinchoninic acid at a wavelength of 562 nm. The sensitivity of the microBCA assay method is 0.5-20 μg/ml protein. The concentration of total protein in any of the studied preparations of plasmid DNA did not exceed the norm.

The content of bacterial lipopolysaccharide in plasmid DNA preparations was determined using a gel-thrombus variant of the LAL-test, with a sensitivity of >0.25 EU/ml (ToxinSensor, GenScript, USA). A lysate of horseshoe crab Limulus Polyphemus amebocytes served as a LAL-reagent. LAL-reagent reacts specifically with bacterial endotoxins, as a result of enzymatic reaction there is a change in the reaction mixture, proportional to the concentration of endotoxin. Results were assessed by the presence or absence of a dense thrombus at the bottom of a tube by inverting the tube. Gel-thrombus was not formed in the study of the sample diluted 10 times, for preparations of all the plasmid DNA obtained, i.e. when the sensitivity of the method is 2.5 EU/ml, which, given the concentration of the plasmid DNA in the sample, confirms a valid indicator of purification from endotoxins.

### Example 3. Determination of the Analgesic Effect of the Plasmid DNA Encoding Modified Beta-Endorphin

3.1. Hot Plate Test

A study of the analgesic activity of the plasmid DNA encoding a modified beta-endorphin was conducted using the test “hot plate”.

Hot Plate Test was carried out to measure the threshold of acute pain sensitivity and potential analgesic effect of the studied preparations of plasmid DNA [Valdman A. V., Ignatov Yu. D. Central mechanisms of pain.—L.: Science.—1976]. The test is basic for the study of analgesic activity, it is used to identify the analgesic active compounds.

After placing the animal on a hot surface, when the threshold of pain sensitivity is reached, the anxiety motive reactions of the animal are observed: a flick of the paws, licking the pads and jumping. In this test, the latent time from the moment of placing the animal on a hot surface to the first licking of the paws was taken into account. Using this method, the following indicators are determined: analgesic activity of the test object, peak time of analgesia, duration of analgesia.

Mice of line BALB/C, females, weighing 15 to 22 grams, age 18 weeks were used in the study. Experimental and control groups of animals were formed in the study, each group consisted of 3 mice. Analgin (50 mg/kg), morphine hydrochloride (10 mg/kg), and pcDNA3.1(+) plasmid DNA encoding native beta-endorphin (YGGFMTSEKSQTPLVTLFKNAIIKNAYKKGE) with the secretory sequence without codon-optimization for expression in mammalian cells were used as a positive control.

5 mg/kg of plasmid DNA were administered.

An appliance Hotplate Meter (Hotplate Analgesia Meter, Columbus Instruments, USA) was used.

Prior to the study the test systems were allowed to acclimatize for 10 minutes in the room for the study. The animals were used once, since the repeated placement of the animal on a thermostatted plate causes an immediate reaction to the touch of the surface. Thermostat temperature was set at 55° C. After the injection of the test substance, the animal was carefully placed on the heating plate and at the same time the “start” button was pressed on the appliance panel. Latency time of licking of the front and rear paws (from the moment of placing the animal on the surface of the appliance before the first licking) was noted. After that, the “stop” button was pressed and the animal was removed from the hot surface. Other behavioral reactions were ignored. To reduce the probability of thermal damage of paw pads, the maximum experiment time did not exceed 60 seconds. To determine the peak time of analgesic action of the preparation, the latent time of licking the fore and hind paws was measured in the control group (saline) (point 0) and 2 h, 12 h, 24 h after administration of the preparation in the tested groups. The surface of the appliance was wiped with a napkin moistened with a disinfectant (0.5% chlorhexidine-digluconate with 70% ethanol) before placing another animal on it [Guidelines for Trainees. Faculty of Pharmacy of GOU VPO MMA named after I. M. Sechenov of Roszdrav.—Moscow.—2006].

The analysis of data of determination of analgesic activity of a preparation was performed. For the plasmid DNA, which has shown the best result, ED50 was determined, i.e. the dose necessary for the demonstration of 50% analgesic activity of the preparation.

Preparations of all studied plasmid DNAs encoding modified beta-endorphin exhibit a significant analgesic effect not inferior in efficiency to morphine and exceeding analgesic activity of analgin, as well as of plasmid DNA encoding native beta-endorphin. On average, a small level of analgesia was observed 2 hours after the plasmid administration, then the analgesia increased, and after 12 and 24 hours the same high level of analgesia was observed, indicating the maintenance of the effect.

In the experiment using different doses of plasmid DNA, which has shown the best results in the previous experiment (pVAX1, with the second variant of the secretory sequence used, and the fragment mediating binding to TLR9 placed next to the origin of replication), the dose-dependent effect of analgesia was demonstrated in the range of 0.2 up to 10 mg/kg. At the same time, after 12 hours, the indicators when using such plasmid DNA in a minimum concentration of 0.2 mg/kg were almost identical to those in the group using morphine hydrochloride 10 mg/kg. After 24 hours, the indicator in the group of plasmid DNA in the minimum concentration exceeded that of the group using morphine hydrochloride 10 mg/kg, almost in 2.5 times. The effect of analgin was observed only after 2 h after the introduction of the analgesic, the response after 12 h and 24 h was similar to that in the control group. When using the plasmid DNA mentioned above, at a concentration of 5 mg/kg and 10 mg/kg, a significant increase in the latent period was observed, compared with the group with a minimum concentration of the plasmid DNA, with a small difference between these two groups.

3.2. Study of Beta-Endorphin Concentration in Mouse Serum

Since the change in the concentration of beta-endorphin in the blood plasma can serve as a criterion for evaluating the effectiveness of anesthesia [Beta-endorphin—a marker of the effectiveness of anesthesia in acute pain and chronic pain in cancer patients/Z. V. Pavlova [and others]//Problems of clinical medicine.—2007.—N1.—P. 36-40.—ISSN 1817-8359], a study of the concentration of beta-endorphin in the serum of the studied mice after the introduction of the developed plasmid DNA was conducted using ELISA Kit for Beta-Endorphin (bEP) Mus musculus (Mouse) CEA806Mu (Life science Inc.). The level of beta-endorphin rose significantly with the introduction of the developed plasmid DNA, despite the fact that with the introduction of plasmid DNA without inserting the targeted gene and in the control group, a significant increase in the level of beta-endorphin was not observed throughout the experiment. In the group where plasmid DNA encoding native beta endorphin was used, the level of beta endorphin was lower than in the experimental groups.

Thus, there were demonstrated the possibility of creating various variants of plasmid DNA encoding modified beta-endorphin, their production using a bacterial producer, as well as the analgesic effect of such plasmid DNA, in any of the variants that match the specified criteria, even in a minimal concentration of 0.2 mg/kg (many times less than morphine hydrochloride). A higher level of beta-endorphin synthesis was also shown, as well as a greater efficiency than that of plasmid DNA encoding native beta-endorphin and not containing a fragment causing TLR9 binding. The safety of using such plasmid DNA was also demonstrated: the animals did not die, no side effects were observed.

1-3. (canceled)

4. A plasmid DNA for transient expression in mammalian cells comprising prokaryotic elements, a replication origin and a marker gene, and eukaryotic elements, a strong promoter, a leader sequence of mRNA and also regulatory sequences for these elements, at least one site for cloning a gene of interest and at least one site for annealing of at least one single primer for analysis of plasmid DNA composition, a fragment set forth as SEQ ID NO: 1 repeated ten times sequentially, located between any two elements of the plasmid DNA, and a polynucleotide comprising a fragment encoding a heterologous secretory sequence and a fragment encoding a modified beta-endorphin set forth as SEQ ID NO: 2, the polynucleotide being codon-optimized for expression in mammalian cells, and a termination sequence.

5. A producer of the plasmid DNA according to claim 4, based on the bacterial cell.

6. An analgesic agent comprising the plasmid DNA according to claim 4 in an effective amount and a pharmaceutically acceptable excipient, for mammals.

7. An analgesic agent according to claim 6 for a human.
